(19) United States
(12) Patent Application Publication
(10) Pub. No.: US 2005/0244450 A1
(43) Pub. Date: Nov. 3, 2005

(54) Heat-treated implantable bone material

(76) Inventor: A. Hari Reddi, El Macero, CA (US)

Correspondence Address: HARNESS, DICKEY & PIERCE, P.L.C., P.O. BOX 828, BLOOMFIELD HILLS, MI, 48303, US

(21) Appl. No.: 10/835,519

(22) Filed: Apr. 28, 2004

(51) Int. Cl. A61F 2/28

(52) U.S. Cl. 424/423; 623/23.63; 435/378

(57) ABSTRACT

A method of making an implantable bone material and a product made by the method. The method includes forming a mixture of water and demineralized bone powder comprising collagen, and heating the mixture under non-denaturing conditions.

The present invention relates to compositions and methods for bone repair or growth. In particular, such compositions include those comprising demineralized bone.

Generally, the skeletal structure of mammalian anatomy is strong enough to maintain its rigidity and structure through a given amount of force or use. Nevertheless, the skeletal structure may be weakened or damaged for any of a variety of reasons, including disease, trauma, age, congenital defects, and surgical procedures. A variety of bone repair materials have been described to aid in the repair or reconstruction of such bone defects.

Among the materials that have been suggested for bone repair or reconstruction is bone allograft. One form of bone allograft is demineralized bone, which is typically formed through chemical treatment of bone so as to remove most or all of its mineral content. The remaining demineralized bone matrix (“DBM”) consists of the native collagen structure of the bone, along with naturally-occurring growth factors, e.g., bone morphogenetic proteins (“BMPs”). DBM is both osteoinductive and osteoconductive. Osteoconduction is the promotion of differentiated bone-forming cells growth or infiltration into the DBM from the subject in whom the DBM is implanted. Osteoinduction is the promotion of new bone-forming cell production, from non-differentiated cells, in and around the implanted DBM matrix.

DBM is typically provided for clinical use from “bone banks,” which harvest bone from human cadavers (donated and managed according to proper ethical and legal standards). The bone undergoes physical processing (such as grinding or shaping), and is then demineralized to form DBM. Because the bone may be harvested and processed in advance of its use, it is frequently dried (e.g., by lyophilization) and packaged under sterile conditions, for storage and shipping to the clinical site.

In practice, however, it has been found that there is significant variability in the bone-building activity of DBM prepared by such techniques. The source of such variability is not understood, but (without limiting the composition, mechanism or utility of the present invention) may be due to differences in the bone taken from source cadavers and variability in the demineralization and other processing of the bone prior to use. In some cases, production lots of DBM are tested to determine their bone building activity (in particular, osteoinductive activity), and samples found to have little or no activity are rejected for use as implant materials.

### SUMMARY

The present teachings provide methods of making an implantable bone material. The methods include forming a mixture of water and demineralized bone comprising collagen, and heating the mixture under non-denaturing conditions, preferably effective to physically alter the collagen structure. The present teachings also provide products made by such methods.

The present teachings also provide an implantable bone material composition. The composition includes a demineralized bone material and a pharmaceutically-acceptable carrier, wherein the demineralized bone material is made by a process of consisting essentially of demineralizing bone powder and mixing said powder with water, and heating the mixture under non-denaturing conditions, preferably effective to physically alter the collagen.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DESCRIPTION

The following definitions and non-limiting guidelines must be considered in reviewing the description of this invention set forth herein.

The headings (such as “Introduction” and “Summary,”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof.

The citation of references herein does not constitute an admission that those references are prior art or have any relevance to the patentability of the invention disclosed herein. All references cited in the Description section of this specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations of the stated features. Specific Examples are provided for illustrative purposes of how to make, use and practice the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the word “include,” and its variants, is intended to be non-limiting, such that recitation of items in a list is not to the exclusion of other like items that may also be useful in the materials, compositions, devices, and methods of this invention.

The present invention provides bone derived compositions for the treatment of bone defects in humans or other animal subjects. Specific materials to be used in the invention must, accordingly, be biocompatible. As used herein, such a “biocompatible” component is one that is suitable for use with humans and/or animals without undue adverse side effects (such as toxicity, irritation, and allergic response) commensurate with a reasonable benefit/risk ratio.

In various embodiments compositions of the present invention are made by a process comprising:
(a) forming a mixture of water and demineralized bone comprising collagen; and
(b) heating the mixture under non-denaturing conditions..

As referred herein “demineralized bone” refers to bone material from which a substantial portion of naturally-occurring minerals have been removed. Preferably the mineral content of the demineralized bone powder is less than about 20%, optionally less than about 10%, optionally from about 0% to about 5%, optionally from about 0% to about 2%, optionally less than about 0.5%. (As referred to herein, all percentages are by weight unless otherwise specified.)

Such bone material includes bone powders and other bone constructs such as cubes, rods, dowels, pins, disks and other formed devices. In one embodiment, the bone construct has been entirely demineralized. In another embodiment, the bone construct has been demineralized in one aspect of its structure, e.g., by demineralizing the surface of the construct. In one embodiment, the bone material comprises bone powder, comprising particulates that are less than about 1500 microns in size. In various embodiments, the bone particles range from about 50 microns to about 1000 microns, from about 75 to about 800 microns, or from about 150 to about 600 microns. Depending on the desired composition, particles may be of a variety of sizes.

In one embodiment, bone powder is obtained from animal sources (i.e., for xenogenic implantation in a human subject) such as cows and pigs. In another preferred embodiment, bone is obtained from human cadavers (i.e., for allogenic implantation in a human subject), following appropriate ethical and legal requirements. Such human bone material is available from a variety of tissue banks.

The bone from which the DBM is produced may comprise cortical bone, cancellous bone, or a combination thereof. Cancellous bone is available in a range of porosities based on the location in the body from which the bone is harvested. Highly porous cancellous bone may be harvested from various areas such as the iliac crest, while less porous bone may be harvested from areas such as the tibial condyle femoral head, and calcaneus. Cortical bone may be obtained from long bones, such as the diaphyseal shaft of the femur and tibia. A preferred implant comprises cortical bone.

The bone material is demineralized using any of a variety of methods, including those known in the art using acids, chelating agents and electrolysis. Preferred chemical treatments include those using hydrochloric acid, ethylene diamine tetraacetic acid (EDTA), peracetic acid, or citric acid. Demineralization techniques among those useful herein are described in K. U. Lewandrowski et al., “Kinetics of cortical bone demineralization: controlled demineralization—a new method for modifying cortical bone allografts,” J Biomed. Mater. Res., 31:365-372 (1996); K. U. Lewandrowski, et al., “An electron microscopic study on the process of acid demineralization of cortical bone,” Cal. Tiss. Int., 61:294-297 (1997); and K. U. Lewandrowski, et al., “Improved osteoinduction of cortical bone allografts: a study of the effects of laser perforation and partial demineralization,” J Orthop. Res., 15:748-756 (1997); and Reddi et al., “Biochemical sequences in the transformation of normal fibroblasts in adolescent rats,” Proc. Nat. Acad. Sci., 69 pp. 1601-5 (1972).

The demineralization treatment provides a DBM comprising insoluble collagen and other non-collagenous proteins such as bone growth factors including bone morphogenetic proteins (BMPs). In one embodiment, the DBM is made by a process consisting essentially of demineralizing the bone, such that, prior to the heating step of the present process, the DBM has not undergone treatments that destroy the chemical composition of collagen, or denature bone growth factors within the DBM. In one embodiment, the heating step is performed on a bone composition consisting essentially of the DBM. Such compositions do not comprise added osteogenic proteins. As referred to herein, “osteogenic proteins” are proteins that are capable of producing a developmental cascade of cellular events resulting in endochondral bone function. Such osteogenic proteins are those referred to in the art as osteogenic proteins, osteoinductive proteins and bone morphogenetic proteins. The DBM may, however, include non-osteogenic proteins and active materials, such as those selected from the group consisting of synthetic and recombinant growth factors, growth factor mimetics, morphogens, and plasmid and viral vectors. Products made using such compositions may further comprise added osteogenic proteins and such non-osteogenic proteins and active materials added to the irradiated DBM after irradiation.

In one embodiment, the bone powder is subjected to defatting/disinfecting and acid demineralization treatments. A preferred defatting/disinfectant solution is an aqueous solution of a lower alcohol, such as ethanol. Preferably, the defatting/disinfection solution contains at least about 10% to 40% water (i.e., about 60% to 90% defatting agent such as alcohol). Preferably, the solution contains from about 60% to about 85% alcohol. Following defatting, the bone is immersed in acid or chelating agent over time to effect demineralization. The concentration of the acid or chelating agent in such demineralization operation is preferably about 0.5 N to about 1.0 N, with demineralizing time being from about 2 to about 12 hours under ambient conditions.

The forming a mixture of water and the demineralized bone (herein “DBM mixture”) may be produced from the demineralization process, or may comprise adding water to the demineralized bone. In embodiments comprising DBM powder, the powder is admixed with water, such that the powder is thoroughly wetted. In embodiments comprising a DBM construct, such as a surface demineralized bone device, the bone may be immersed in water or otherwise contacted with water such that the demineralized portion of the bone is thoroughly wetted.

In one embodiment, the DBM mixture comprises some or all of the water used in the demineralizing process. In one embodiment, the demineralizing acid solution is used directly in the heating step. In another embodiment, the acid solution is neutralized prior to the heating step. In one embodiment, additional water is added to the demineralizing solution prior to the heating step. In one embodiment, the DBM mixture is made by adding water to dried, or substantially dried, demineralized bone. Preferably, the added water is sterile water.

In various embodiments, the DBM mixture comprises from about 5% to about 20%, optionally from about 9% to about 16% by weight of water. In one embodiment, water is added to the DBM powder in a level of from about 5 to about 10 ml per gram of DBM.

As referred herein, the phrase “heating the mixture under non-denaturing conditions” means temperature and pressure conditions that do not significantly alter the chemical composition of the individual stands of the collagen structure. In one embodiment, the heating is effective to physically alter the collagen in the DBM, i.e., heating which alters the physical structure of the collagen structure or individual strands thereof, without significantly altering the chemical composition of the individual stands of the collagen structure. Under these conditions, heating is, for example, below the temperature that converts collagen into gelatin at a given pressure. In particular, steps that would induce denaturing of the DBM's collagen, such as subjecting the DBM to acid-promoted cleavage, cross-linking the collagen, etc., are excluded under the non-denaturing conditions defined above. Without limiting the composition, utility or mechanism of the present invention, in some embodiments the heating does not effect melting of the collagen (i.e., disruption of the triple helical collagen structure). In other embodiments, the heating melts the collagen.

In one embodiment, heating is effected at ambient pressure (it being understood that ambient pressure may vary according to altitude and weather conditions) Preferably, at ambient pressure, the DBM mixture is heated at a temperature of about 28° C. to 65° C., preferably from about 38° C. to about 60° C., preferably from about 45° C. to about 58° C., preferably from about 50° C. to about 58° C. for a period of from about 1 to about 24 four hours, preferably for from about 2 to about 15 hours, preferably from about 3 to about 10 hours. In one embodiment, the DBM mixture is heated at a temperature of about 56° C. for about 6 hours.

In various embodiments, the present invention provides bone materials formed from DBM that is prepared and treated to induce and/or increase bone growth activity in the subject to whom the DBM is implanted. Such bone growth activity includes osteoinduction. In one embodiment, the osteoinductivity of a given human DBM composition is increased. In one embodiment, the present invention provides a method for improving the osteoinductivity of demineralized bone, comprising:
(a) forming a mixture of water and human demineralized bone having little or no osteoinductivity; and
(b) heating the mixture under non-denaturing conditions, preferably effective to physically alter the collagen.
   In one embodiment, the present invention provides methods for producing demineralized bone compositions having uniform osteoinductivity, the method comprising:
(a) obtaining bone portions from a plurality of human donor subjects;
(b) forming a mixture of water and demineralized bone powder made from the bone portions; demineralizing the bone portions to produce demineralized bone portions; and
(c) heating the mixture under non-denaturing conditions, preferably effective to physically alter the collagen.
   In such methods, DBM portions from different human donors that have different levels of osteoinductivity, including little or no osteoinductivity, can be normalized by irradiating as above, such that all the DBM portions have a non-zero baseline level of osteoinductivity when implanted. Moreover, the level of osteoinductivity can have a desirable distribution among portions and can be also made to be substantially uniform among portions.

In various embodiments, osteoinductive activity is determined using an accepted in vivo or in vitro measurement of activity. Such methods include in vivo implantation methods with histological analysis, and in vitro assays such as alkaline phosphatase assays and cell proliferation assays. In one embodiment, comprising obtaining bone portions from a plurality of human donor subjects, samples of the portions are tested before and after irradiation. In a preferred embodiment, no such testing is performed.

A suitable test comprises a rat in vivo model. In one such model, approximate 0.3 cc aliquots of the compositions are aseptically packed into 1 cc syringes with the barrel blunt cut at a 45° angle. Male rats (strain Hsd:Rh-rnu) are weighed and randomly assigned to a treatment group. The material is placed in a pocket created between the semi-membranous and adductor muscle group in close proximity to the femur. The material is implanted in each leg. Body weights are recorded prior to implantation, weekly and at termination. Animals are observed daily for general health and detailed examinations for clinical signs of disease or abnormality are conducted at randomization, weekly and at termination. After 28 days, the rats are euthanized and the entire limbs are removed and fixed. The entire implant area, including the femur, is transected above and below the implant area. The harvested tissues are sectioned at multiple levels (proximal, middle and distal implant area), decalcified overnight and histologically processed (embedded, sectioned and stained in hematoxylin and eosin and Toluidine blue).

Following processing, all tissue sections are examined for new bone and new cartilage and evaluated with a microscopic scoring scheme based on the following Osteoinduction Grading Scale:
0 =0% of implant area occupied by new bone
1 =1-25% endochondral ossification and/or new bone covering 1-25% of implant
2 =26-50% endochondral ossification and/or new bone covering 26-50% of implant
3 =51-75% endochondral ossification and/or new bone covering 51-75% of implant
4 =76-100% endochondral ossification and/or new bone covering 76-100% of implant.

In another embodiment, the tissue sections are evaluated according to the following Modified Schwartz System:
0 =no implant
1 =implant present, no new bone or cartilage present
2 =<25% endochondral ossification and/or new bone covering <25% of implant
3 =26-50% endochondral ossification and/or new bone covering 26-50% of implant
4 =>50% endochondral ossification and/or new bone covering >50% of implant

The processes of the present invention optionally comprise other steps, including physical processing of the DBM, sterilization, and packaging. Such additional process steps may be performed before or after heat treatment, as appropriate. The demineralization process may produce a particulate or other product, which may be further ground to a substantially fine particulate in the form of DBM powder. It will be understood that the DBM powder may be formed in any appropriate manner as required in a particular clinical application or procedure. Sterilization includes irradiation as discussed below or chemical sterilization techniques (such as using ethylene oxide). Packaging includes methods suitable for convenient storage, handling or transport of the composition after preparation and before use. In one embodiment, such packaging includes lyophilization of the composition to remove substantially all water.

The heat-treated DBM powder or article made thereof may be further enhanced for osteoinduction after or before heat treatment by irradiating at cool temperatures, as disclosed in co-filed Patent Application Ser. No. ______, Reddi, “Irradiated Implantable Bone Material”, incorporated herein by reference. In one such process, the heat-treated DBM powder or product is irradiated at temperatures less than about 0° C. with gamma radiation at a dose of from about 0.5 Mrad to about 15 Mrad, preferably from about 1 to about 10 Mrad, preferably from about 1.5 to about 5 Mrad. Such irradiation may be conducted at a sterilizing dose for sterilizing of the final product, or may be conducted prior to manufacture of the final product.

The compositions of the present invention may be used in any of a variety of clinical procedures for the treatment of bone defects. As referred to herein such “bone defects” include any condition involving skeletal tissue which is inadequate for physiological or cosmetic purposes. Such defects include those that are congenital, the result of disease or trauma, and consequent to surgical or other medical procedures. Specific defects include those resulting from bone fractures, osteoporosis, spinal fixation procedures, and hip and other joint replacement procedures. The DBM composition may be used to assist in bone reconstruction, soft tissue fixation and the like, either as-is, as a wet or dry or lyophilized DBM powder, or formulated into putty, sheet, or other DBM product. In various embodiments, the composition is combined with another material, composition, or device. Suitable products comprising a composition of the present invention include those disclosed in U.S. Pat. No. 5,348,788, White, issued Sep. 20, 1994; U.S. Pat. No. 5,455,100, White, issued Oct. 3, 1995; and U.S. Pat. No. 5,487,933, White, issued Jan. 30, 1996; U.S. Pat. No. 6,576,249, Gendler et al., issued Jun. 10, 2003; and PCT Patent Publication WO 03/051240, Schwardt et al., published Jun. 26, 2003.

In one embodiment, a composition of the invention is formed into a product/article or into a particular shape, either with the use of a carrier, or a binder, or simply by shaping the powder into a selected shape in a pocket, recess, bore or other receiving surface of the implantation site. The article or shape could be a sheet, a disc or other flat plate, an elongated member, such as a bar or rod, a bone-shaped member, or any other member shaped to be received in a bore of a bone or other body portion, including a plug, a ball or other article shaped for filling a cavity of the same shape. The article can also be a two- or three-dimensional porous structure with holes or depressions and protrusions, which can be formed by using appropriate mesh sheets as discussed above.

The material of the invention may also be mixed with a biocompatible carrier. Such carriers include saline, hyaluronic acid, cellulose ethers (such as carboxymethyl cellulose), collagen, gelatin, autoclaved bone powder, osteoconductive carriers, and mixtures thereof. Osteoinductive carriers include allograft bone particles, other demineralized bone matrix, calcium phosphate, calcium sulfate, hydroxyapatite, polylactic acid, polyglycolic acid and mixtures thereof. Other carriers include blood, monosaccharides, disaccharides, water dispersible oligosaccharides, polysaccharides, low weight organic solvents, including glycerol, polyhydroxy compounds, such as mucopolysaccharide or polyhyaluronic acid and various aqueous solutions, as described in U.S. Pat. Nos. 5,290,558; 5,073,373; 5,314,476; 5,507,813; 4,172,128; and 4,191,747. In one embodiment, the carrier comprises gelatin. In one embodiment, the carrier is a bone-derived material. One such bone-derived material is made by mixing DBM with water or saline, and heating under denaturing conditions to form a viscous composition. Preferably, the DBM mixture is heated under autoclaving conditions, at a temperature of at least about 85° C. and pressure of at least about 15 psi, for at least about 1 hour. Optionally, the autoclaving is at a temperature of from about 85° C. to about 100° C. and a pressure of from about 15 psi to about 90 psi, for from about 1 to about 8 hours. Such compositions are described in U.S. Pat. No. 6,576,249, Gendler et al., issued Jun. 10, 2003.

In one embodiment, the composition is positioned in a selected position or orientation for implantation, which includes a predefined shape. For example, the heat-treated DBM powder may be positioned in a bore, either with or without a binder or adhesive, such as bone cement, and the bore may form the shape which the DBM powder will take, such that the DBM powder is not preformed in a selected shape. Therefore, forming the DBM powder into a selected shape before implantation will be understood to be optional and not necessary.

The present invention is further illustrated through the following non-limiting example.

### EXAMPLE

In a method of this invention, bone is harvested from a single human donor. The bone is ground to and fractionated to have a particle size of from about 150 to 600 microns. The powder is lipid extracted in ethanol, and demineralized with 0.6 N HC1 for about 12 hours. The DBM powder is mixed with sterile water in the ratio of 1 gram of DBM powder to five ml of sterile water. The mixture is sealed in vials and packaged in foil for treatment or for control. The treatment packages are placed in a water bath at 56° C. for six hours.

The osteoinductivity of the samples is then tested using the rat osteoinduction model described above. The implants of the heat-treated DMB mixture are associated with more new cartilage and more new bone formation than the control implants of the untreated DMB mixture. Specifically, based the Modfied Schwartz Scheme, 75% of the treated implants have a grade score of 3 and 25% a grade score of 2. In contrast, 50% of the untreated implants have a grade score 3 and 50% a grade score of 2.

Further to the above example, samples of the composition that were heated at 56° C. are lyophilized. A sample is then mixed with blood obtained from a human subject undergoing a hip replacement procedure to form a material having a putty-like consistency. The material is then implanted at the surgical site to fill voids around the site of the implant. Radiographic images of the surgical site one month after surgery reveal significant bone building at the site of the implant.

The examples and other embodiments described herein are exemplary and not intended to be limiting in describing the full scope of compositions and methods of this invention. Equivalent changes, modifications and variations of specific embodiments, materials, compositions and methods may be made within the scope of the present invention, with substantially similar results.

1. A method of making an implantable bone material, the method comprising:
(a) forming a mixture of water and demineralized bone comprising collagen;
and
(b) heating the mixture under non-denaturing conditions.

2. A method according to claim 1, wherein heating is conducted under conditions effective to physically alter the collagen.

3. A method according to claim 2, wherein heating is conducted under ambient pressure, at a temperature from about 45° C. to about 65° C.

4. A method according to claim 3, wherein the temperature is from about 56° C. to about 60° C.

5. A method according to claim 2, wherein heating is conducted for about 1 hour to about 24 hours.

6. A method according to claim 1, additionally comprising removing excess water after heating.

7. A method according to claim 6, additionally comprising irradiating the demineralized bone after removing excess water.

8. A method according to claim 7, wherein said irradiating comprises exposing the demineralized bone to gamma irradiation at a dose of from about 1 to about 10 mRad, while maintaining the demineralized bone a temperature less than about 0° C.

9. A method according to claim 1, wherein the demineralized bone is human demineralized bone.

10. A method according to claim 1, wherein the mixture is irradiated.

11. A product made by a method according to claim 1.

12. A method according to claim 1, wherein heating comprises heating below a temperature effective to create gelatin from collagen.

13. A method according to claim 1, wherein heating comprises heating for about six hours.

14. A method according to claim 1, further comprising lyophilizing the mixture to a dry powder after heating.

15. A method according to claim 13, further comprising dissolving the dry powder in a solution suitable for injection.

16. A method according to claim 1, further comprising:
(a) adding a biocompatible carrier after heating; and
(b) forming the mixture into an article.

17. A method according to claim 15, wherein the article is selected from the group consisting of a sheet, an elongated member, a plate, a plug, a ball, and a porous member.

18. A method according to claim 1, further comprising:
(a) adding a biocompatible carrier after heating; and
(b) forming the mixture into formable putty.

19. A method according to claim 1, further comprising:
(a) after heating maintaining the mixture at a freezing temperature; and
(b) irradiating the mixture.

20. A method according to claim 18, wherein maintaining the mixture at a freezing temperature comprises freezing at temperature below about 0° C.

21. A method according to claim 18, further comprising packaging the mixture in dry ice.

22. A method according to claim 18, wherein irradiating comprises irradiating at a dose of about 1 to 10 Mrad.

23. An implantable bone material made by a method according to claim 18.

24. A method according to claim 1, wherein the demineralized bone powder further comprises non-collagenous proteins.

25. A method according to claim 1, wherein forming a mixture of water and demineralized bone powder comprising collagen, comprises forming by a process consisting essentially of demineralizing bone powder and mixing the demineralized bone powder with water.

26. An implantable bone material composition, comprising:
(a) a demineralized bone material; and
(b) a pharmaceutically-acceptable carrier, wherein the demineralized bone material is made by a process comprising:
(i) forming a mixture of water and demineralized bone powder comprising collagen, by a process consisting essentially of demineralizing bone powder and mixing said powder with water; and
(ii) heating the mixture under non-denaturing conditions.

27. A method according to claim 26, wherein heating is conducted under conditions effective to physically alter the collagen.

28. A method according to claim 27, wherein heating is conducted under ambient pressure, at a temperature from about 45° C. to about 65° C.

29. The composition of claim 26, wherein the composition is lyophilized.

30. The composition of claim 26, wherein the carrier comprises a bone-derived material.

31. The composition of claim 28, wherein the carrier comprises gelatin.

32. The composition of claim 28, wherein the bone-derived material comprises autoclaved bone powder.
